(19) United States
(12) Patent Application Publication
(10) Pub. No.: US 2025/0002902 A1
(43) Pub. Date: Jan. 2, 2025

(54) SYSTEMS, COMPOSITIONS, AND METHODS FOR UNIQUELY IDENTIFYING AND ANALYZING NUCLEIC ACID MOLECULES

(71) Applicant: Fluid Discovery, San Francisco, CA (US)

(72) Inventor: Adam Abate, San Francisco, CA (US)

(21) Appl. No.: 18/808,728

(22) Filed: Aug. 19, 2024

Related U.S. Application Data
(60) Provisional application No. 63/585,101, filed on Sep. 25, 2023.
(60) Provisional application No. 62/520,365, filed on Jun. 15, 2017.

(51) Int. Cl. C12N 15/10

(52) U.S. Cl. CPC C12N 15/1065

(57) ABSTRACT

Provided herein are systems, compositions, and methods for uniquely identifying and analyzing nucleic acid molecules. In particular, provided herein are systems, compositions, and methods employing modified base identifiers to generate a unique pattern in individual nucleic acid molecules and analyzing the modified molecules, or copies thereof, to identify or differentiate the molecules.

### CROSS-REFERENCE TO RELATED APPLICATIONS

The present application claims priority to U.S. Provisional Patent Application Nos. 63/585,101, filed Sep. 25, 2023; and 62/520,365, filed Aug. 18, 2023, each of which are incorporated by reference in its entirely.

### FIELD

Provided herein are systems, compositions, and methods for uniquely identifying and analyzing nucleic acid molecules. In particular, provided herein are systems, compositions, and methods employing modified base identifiers to generate a unique pattern in individual nucleic acid molecules and analyzing the modified molecules, or copies thereof, to identify or differentiate the molecules.

### BACKGROUND

The detection and analysis of nucleic acid molecules in complicated sample types, including biological and environmental samples, remains a challenge. Molecular biology techniques, including nucleic acid amplification and sequencing, for example, can generate complicated mixtures of molecules, including some that may incorporate errors, making it challenging to determine the identity or source of specific molecules are sequences. Improved systems, compositions, and methods are needed.

### SUMMARY

Provided herein are systems, compositions, and methods for uniquely identifying and analyzing nucleic acid molecules. In particular, provided herein are systems, compositions, and methods employing modified base identifiers to generate a unique pattern in individual nucleic acid molecules and analyzing the modified molecules, or copies thereof, to identify or differentiate the molecules.

For example, in some embodiments, provided herein are methods for uniquely identifying a target nucleic acid molecule using modified base identifiers (MBIs) comprising: a) modifying a molecule or plurality of molecules or its/their copy/copies with base modifications (e.g., randomly incorporated base modifications), wherein a generated pattern of modified bases are unique or substantially unique to the molecule or each individual member of the plurality of molecules; and b) reading the modified molecule, or a copy of part or all thereof, and determining the locations and/or types of modified bases to differentiate the modified molecule from other molecules (e.g., of identical original sequence or from a sample containing multiple identical or different nucleic acid molecules).

In some embodiments, the target nucleic acid is RNA and the copying comprises reverse transcription. In some embodiments, the target nucleic acid is DNA. In some embodiments, the modified nucleotide bases comprise methylated nucleotides. In some embodiments, the modified nucleotide bases are mixed at a ratio with respect to unmodified bases of the same nucleotide base, for example, A for A*, G for G*, and so on. In some embodiments, the multiple modified nucleotides of different base (e.g., A, T, G, C) and/or different modification type are used to control or expand an encoding scheme. In some embodiments, the frequency of modified bases is controlled by controlling the ratio of modified to non-modified base concentrations during the copying step so as to ensure a desired number of random incorporations during the copying step to allow differentiation of targets of similar sequence to the desired degree (e.g., selecting a ratio that statistically generates at least one, at least two, at least three, at least four . . . modified base in any given molecule).

In some embodiments, the method is combined with other tag sequences that uniquely identify the molecule, such as cell or sample barcodes or unique molecular identifiers (UMIs).

In some embodiments, the target is modified prior to copying, resulting in a base transformation that can be used in combination with other approaches to produce an MBI, such as the deamination of Adenine to Inosine, resulting in a Guanine in the cDNA, therefore generating an A-C (G) transformation.

In some embodiments, the number of resulting MBI combinations is related to N!/n! (N-n)!, where N is the number of modifiable bases in the target, and n is the number of modified bases. A typical read for a transcript has ˜25 modifiable bases, so incurring 1 modified base yields 25 unique sequences, while incurring 3 modified bases yields 13,800. The total number permutations is 2\hat{ }25 =˜34M. Thus, 3 modified bases exceeds in bit depth what is typically achieved with a 6 base UMI (4096). If different modifications occur (e.g., A to T and, in parallel, G to C), then the results multiply Nat!/nat!(Nat-nat)!*Ngc!/ngc!(Ngc-ngc)!.

In some embodiments, the modified bases may include Thymine Glycol Formation (T to G Conversion), 8-Oxoguanine Formation (G to T Conversion), Alkylation of Guanine (G to A Conversion), Uracil in DNA (Originally from Thymine Deamination or incorporated as a base), Hydroxymethylation and Further Oxidation (C to T Conversion), Deamination (C to U Conversion, leading to C to T), Hypoxanthine Formation (A to G Conversion), and others. These can be applied to the target molecule or its copy or copies, or incorporated as bases during copying.

In some embodiments, the a degree of randomization is incorporated into the copying step to introduce an MBI, for example, by using error prone enzymes, non-canonical nucleotides, polymerase inhibitors, or altering reaction conditions.

In some embodiments, the reading step comprises swapping the modified base for another base, such as A* for U, G, etc., using methods such as bisulfate transformation, enzymatic transformation, etc.

In some embodiments, the reading step comprises amplifying the copied molecule to produce amplicons while preserving information relating to the locations and/or types of the randomly incorporated bases, for example, by swapping modified bases with unmodified bases (eg, A* for G) according to a prescribed transformation reaction for different unmodified and modified bases.

In some embodiments, the locations and types of modified bases are determined by sequencing the copies or their amplification products and determining the locations of the modified bases in the sequence reads by comparing the read with reads of similar sequence and identifying locations of known conversions.

In some embodiments, the reads of the amplicons are grouped by their unique but conserved modified base spectrum such that all reads for a given original target can be used to generate a consensus sequence that uniquely differentiates it from all other original target molecules of identical starting sequence.

In some embodiments, the unique modified base incorporation, as determined herein, is used to enumerate the number of original target molecules in the sample and/or correct for reaction biases.

In some embodiments, the modified base identifiers are used in conjunction with other tagging schemes, including cell barcoding, UMIs, etc., to perform quantitative single cell gene expression profiling.

In some embodiments, modified base identifiers are used to label the full target nucleotide, allowing regions far from the 3′ or 5′ ends of the molecules to be uniquely identified (e.g., in contrast, UMIs require a sequence on the end): e.g., Deamination (A to I Conversion, leading to A to G) or Alkylation (A to T or C Conversion).

In some embodiments, duplex sequencing is used to differentiate between modifications on two strands (Watson and Crick strands) of a double stranded molecule.

In some embodiments, copies of the original molecule, or sub-portions thereof, are used to build a consensus sequence of the original molecule for error correction or other purposes. For example, the technique of using modified base identifiers (MBIs) can be used for error correction and specialized sequencing applications across various domains like MRD (Minimal Residual Disease) detection, human genetics, and the study of viral quasi-species such as HIV. In the case of MRD, it's often crucial to detect specific sequences among unmodified variants with high resolution. Traditional techniques like duplex sequencing have facilitated this by sequencing both strands of a duplex and confirming results based on complementarity, thus reducing errors generated during the PCR or sequencing process. MBIs can expand on this concept by allowing the same region to be re-sequenced repeatedly for different PCR products. This helps build a consensus sequence that averages out the sequencing error, enabling the detection of rare sequence variants with high certainty. In the realm of human genetics, sequence variants may comprise structural rearrangements that are challenging to detect due to the extensive size of the human genome and the requirements for mapping sequences onto the human reference. Traditional methods like long-read sequencing can be labor-intensive for sequencing through such variants. MBIs offer an approach to concatenate multiple reads together for the same amplicons that have been amplified through PCR. This is particularly useful for complex regions like tandem repeat areas, offering a streamlined approach to sequencing. Studying viral quasi-species poses challenges due to sequence variants being difficult to quantify. The inability to build consensus sequences for each variant in the sample arises from the overlapping sequences of the viruses that share substantial homology. The use of MBIs can overcome this challenge, providing a more precise approach to understanding the variations among viral strains. The application of this technology opens up new avenues for research and clinical studies across diverse fields of genetic science.

In some embodiments, provided herein are methods for introducing modified base identifiers, comprising: reacting a target molecule with a reactant that introduces at least semi-random base modifications into the target or its copies; and using the unique spectrum of base modifications to differentiate targets of otherwise identical sequence. In some embodiments, the reaction comprises a methylase enzyme that randomly methylates bases in the target, thereby generating a unique signature of random base modifications that can be used for target differentiation according to the methods described herein.

In some embodiments, the methods employ natural bases that are converted into other bases in an at least semi-random way, such as base editors, like cytidine deaminase, which converts C->U directly.

In some embodiments, provided herein are methods for accurate single cell gene expression profiling with MBIs. For example, in some embodiments, provided herein are methods for quantitative single cell gene expression profiling using modified base identifiers (MBIs) comprising: a) creating a cDNA copy of mRNA by reverse transcription, in which methylated cytosines are included in the reaction at a fixed proportion to unmethylated cytosines, thereby generating for each cDNA of each mRNA molecule a unique methylated cytosine spectrum; b) using bisulfate conversion to transform the unmethylated cytosines to uracils, leaving the methylated cytosines unchanged; c) copying the modified cDNA (e.g., using PCR) such that Uracils are replaced with Thymines; d) sequencing the products of step (c), e) clustering reads based on their sequence; and f) using the unique locations of the Thymines and Cytosines (MBIs) to differentiate mRNA molecules of original identical sequence and thereby more accurately enumerate the original mRNA molecules of identical sequence despite biases that may be incurred through amplification or other processes. In some embodiments, the method is combined with single cell barcoding.

In some embodiments, provided herein are methods for single molecule long read sequencing by shotgun methods. For example, in some embodiments, provide herein are methods for sequencing long molecules in otherwise identical sequence, comprising: a) labeling nucleic acid molecules with MBIs; b) amplifying the molecules, preserving the MBIs in some of the copies; c) randomly fragmenting and sequencing the amplicon fragments, such that MBIs remain in the fragments and, in some case, the same MBI region exists within different fragments; and d) using the similarity of the MBI regions in different fragments to associate them together to generate a longer read.

In some embodiments, provided herein are methods for sequencing single cells at high throughput, comprising, a) labeling the nucleic acids of at least one cell with a tag sequence relating the cell identity; b) labeling the nucleic acids of said cell with MBIs so as to differentiate between nucleic acids of otherwise identical sequence from said cell; and c) analyzing the tag, nucleic acid sequence, and MBI so as to accurately identify the nucleic acids of single cells. In some embodiments, the cells are partitions and the tag sequences relating cell identity are introduced by combinatorial labeling using permeabilization to facilitate incorporation through cell membranes (e.g., Parse/Scale method). In some embodiments, the cells are partitioned in compartments (e.g., droplets or wells), such that the tag sequence is substantially unique to the partitions (e.g., droplet method).

Also provided herein are compositions (e.g., kits, reactions mixtures, sets of reagents, etc.) that are useful, necessary, or sufficient to carry out any of the methods described above or herein. Kit may comprise packaging, instructions, storage containers, instruments, and/or computations devices. In some embodiments, provided herein computational devices and/or software configured to instruct one or more devices to conducts steps of the methods herein and/or to collect, analyze, or report (e.g., display) data (e.g., sequences, error-corrected sequences, enumerated sequences or cells, sample identity, diagnostic results, etc.) generated by the methods.

### DESCRIPTION

In some embodiments, nucleic acids are modified, or copied or amplified, in a manner that incorporates modified bases or sequences into the molecules such that the modified molecules provide a unique or substantially unique molecule within a sample. For example, where the nucleic acid of interest is an RNA molecule, by including a mixture of normal and chemically modified (e.g., methylated) nucleotides in a reverse transcription (RT) reaction, it is possible to achieve random incorporation of the methylated bases alongside their unmodified counterparts. This random incorporation of methylated bases generates a unique signature or “spectrum” on each cDNA molecule. The likelihood of methylated base incorporation at any given position is controllable by adjusting the relative proportions of modified and unmodified nucleotides in the reaction. By creating a unique pattern of methylation across each cDNA molecule, individual transcripts can be distinguished from one another. In some embodiments, in the subsequent step the methylated bases are swapped for a different base, such as Thymine (T).

This swap can be achieved through a specific chemical or enzymatic reaction that selectively targets the methylated bases without affecting the unmodified ones. This unique signal or spectrum is then used to distinguish different cDNAs, providing a powerful tool for exploring RNA populations with unprecedented resolution and specificity. Similar approaches can be used to modify DNA molecules. In some embodiments, modifications are made by incorporating modified nucleotides in a copying or amplification reaction. In other embodiments, bases native to the nucleic acid molecule are chemically modified.

The overall concept of a molecular modified base identifier (MBI) revolves around utilizing random chemical modifications on the target sequence or the first few copies of a molecule, which can then be identified later, for example, by sequencing. One example of this approach is incorporating methylated bases during the reverse transcription step that then get transformed into, for example, Thymines. This random incorporation comprises a modified base identifier, but numerous other scenarios or methodologies exist to achieve similar results. Different kinds of bases can be incorporated that lead to transformations, such as changing an A to T, T to G, G to C, and so on. Alternatively, the natural errors of reverse transcription can be used as MBIs, with the frequency of modifications controlled by altering reaction conditions. Another approach is not to replace bases or lead to base transformations during enzymatic copying but to modify the bases so that they ultimately transform into a new base later in the process. For instance, a methylase enzyme can directly methylate DNA, RNA or cDNA, resulting in transformations upon bisulfite or other reactions (e.g., Tet-assisted pyridine borane sequencing, Tet-assisted bisulfite sequencing, APOBEC-coupled sequencing).

In some embodiments, a read for a given RNA or DNA target molecule, with a mostly unchanged sequence, allows that target to be associated with a known gene or source molecule. Once identified, the unique spectrum of modified bases can differentiate between sequences that are similar or otherwise identical, even normalizing for biases that may occur during sequencing reactions. This approach accomplishes what is typically done with a unique molecular identifier (UMI), but with MBIs instead. A useful feature of MBIs is that the identifying sequence or signal accumulates along the entire length of the target molecules, rather than just at one end, as with UMIs. This ability to mark across the full length is valuable, allowing for more robust and continuous tracking of the target molecules throughout the entire sequencing process. Unlike UMIs, where identification information is localized at a specific end, MBIs spread the identification across the whole sequence. This distributed pattern of modification provides higher redundancy and resilience against sequencing errors or potential information loss due to damage. It also allows for more nuanced analyses, as different modifications carry different information. In some embodiments, MBIs are used in combination with UMIs or other exogenous (e.g., index sequences) or endogenous (e.g., fragment or sequence end sequences) markers.

In some embodiments, Modified Base Identifiers (MBIs) are applied directly to molecules without having to copy the molecule. For example, chemical or biochemical enzymatic techniques can be employed to apply modifications to nucleotides on a target in a randomized or semi-randomized manner. Methylase enzymes, which can methylate DNA, can be utilized for this purpose. These random spectrums of molecular bases can then be used as MBIs. This approach offers a novel way to label and identify sequences, utilizing direct modification of the original molecule, and is akin to the method described above, but without the need for replication. It highlights the flexibility and adaptability of the MBI methodology in molecular tracking and analysis.

In some embodiments, the method of Molecular Modified Base Identifiers (MBIs) significantly enhance single-molecule gene expression profiling by offering high quantitativeness of gene counts and the ability to sequence along the entire length of a transcript. For example, in some embodiments, cellular mRNA is first labeled with a cell barcode. Unlike other methods that typically use Unique Molecular Identifiers (UMIs), the approach here employs MBIs to label the transcripts for gene expression profiling. Consequently, each transcript is marked with both a cell barcode and its corresponding MBI. The sequencing is then carried out. Gene expression profiles are estimated, calculated, and normalized based on the cell barcode and MBI methodology. This results in accurate measurements of gene expression profiling for each cell. Essentially, the MBI method achieves what is usually done with UMIs, but with the added benefits related to the breadth and robustness of the tracking across the whole length of the transcripts. The utilization of MBIs in this context offers a more versatile avenue for single-cell sequencing and gene expression analysis.

In some embodiments, the method of using Molecular Base Identifiers (MBIs) is utilized for single molecule long-read sequencing through shotgun methods. This is particularly valuable in cases where one needs to obtain single molecule information from a sample. A challenge arises when molecules in the sample share a significant amount of sequence homology, which can prevent the construction of accurate consensus sequences of the original target molecules. This difficulty arises due to an inability to differentiate between which sequences belong to a given original molecule. Typically, this issue is addressed by using long-read sequencing methodologies like PacBio or nanopore sequencing. However, these approaches have several drawbacks compared to shotgun methods, including being less high-throughput, economical, and accurate. Synthetic long-read sequencing has been developed to overcome these challenges. In this method, molecules are fragmented, and the fragments are labeled with Unique Molecular Identifiers (UMIs). The fragments are then sequenced as short reads using shotgun methods and stitched together based on shared UMIs. Unfortunately, this approach can be complex and require specialized instrumentation such as droplet barcoding or circularization, limiting the data quality, fragment length recovery, and ability to navigate regions with significant repetition.

MBIs provide a robust alternative for achieving single molecule long-read sequencing via shotgun methods. In this approach, MBIs are applied to molecules, creating a unique spectrum on each one. Even if multiple molecules in the sample are identical, the spectrum is applied in a manner that ensures a sufficient density of MBIs. This enables amplicons and fragments to have overlapping MBI regions, allowing the molecules to be stitched together. The process can be executed computationally by finding overlapping regions of MBIs that are identical but reside in different fragments. These can then be connected into a longer fragment. The application of MBIs in this manner addresses the limitations found in other techniques, offering a more versatile and efficient way to achieve long-read sequencing with the benefits of shotgun methods. It leverages the unique properties of MBIs to create a tailored solution for scenarios where traditional methods may fall short.

An example where the technology of using Molecular Base Identifiers (MBIs) is extremely valuable is in the case of HIV sequencing, particularly for examining the HIV reservoir during antiretroviral therapy. In this context, HIV is rare within the sample and may be present at relatively low levels. Furthermore, the majority of the HIV particles are structural and deletion mutants that are nonviable, with typically less than 1% containing the viable virus responsible for the disease. Characterizing these viable viruses, including their frequency and sequences, is a crucial aspect of studying HIV and monitoring the reservoir. This is especially true in response to various intervention strategies. However, this process presents a significant challenge due to the extreme rarity of the viable viruses and the fact that the HIV genomes are so similar. Such similarities make it nearly impossible to efficiently sequence the reservoir and reconstruct the original viral genomes, as they have a high degree of sequence homology. This homology prevents fragments and reads from different HIV viruses in the sample from being distinguishable, since the sequences appear interchangeable. Historically, one of the strategies to address this challenge has been to perform single molecule sequencing on the HIV by using limiting dilution to isolate individual viruses into wells, followed by amplification and sequencing. However, this approach is an extremely laborious process, allowing only the acquisition of a few hundred genomes with significant effort.

MBIs offer a powerful alternative that allows shotgun sequencing of the sample, with all the viruses mixed together, while still being able to reconstruct specific unique HIV genomes. The long-read method described earlier can be applied to label the HIV genomes with a unique MBI spectrum. This labeling allows the fragments to be associated together based on overlapping MBI regions.

By using MBIs, a very efficient and high-throughput strategy for sequencing the reservoir in a single sample can be achieved. It allows for the collection of a large number of genomes very efficiently, providing a substantial advancement in the reservoir characterization process. Such an approach brings more robust capabilities for understanding HIV and can serve as a valuable tool in both research and clinical settings, driving progress in the fight against this complex and persistent viral infection.

The technology described herein, in some embodiments, provides a method for high-throughput single-cell sequencing of DNA, RNA, and/or proteins. Central to this approach are two distinct labeling steps designed for cellular nucleic acids: the first label is unique to each cell, while the second is unique to individual nucleic acids within that cell. This dual-labeling strategy serves two primary purposes:
The first label, often referred to as a cellular barcode, allows for the tracking of all nucleic acids back to their cell of origin. This facilitates the generation of single-cell gene expression profiles.
The second label, designated as the Modified Base Identifier (MBI), differentiates between nucleic acids that have otherwise identical sequences. This enables accurate gene expression measurements within individual cells.

In practical terms, mRNA from a single cell can initially be tagged with a unique cellular barcode. Subsequently, the MBI method is employed to add an additional, distinct label to each nucleic acid molecule originating from that cell.

High-throughput techniques are employed to handle a large number of cells, generating comprehensive datasets of single-cell gene expression profiles. The MBI labels assist in the efficient normalization of these datasets, ensuring accurate results.

### This Dual-Labeling Process is Adaptable to Both Combinatorial Indexing and Compartment Methods

In combinatorial indexing, in some embodiments, cells are fixed and permeabilized, after which the unique cellular barcode is added. The MBI can be introduced either before or after this step, commonly using reverse transcription techniques.
In compartment methods, in some embodiments, such as droplet-based systems, cells are encapsulated in droplets and lysed to release mRNA, which is then tagged with the unique cellular barcode. The MBI is subsequently added, also usually via reverse transcription.

Regardless of the technique employed, the outcome is a nucleic acid library where each molecule is dual-labeled: the first tag indicates its cell of origin, and the second, the MBI, distinguishes it from other identical sequences. This powerful combination of labels aids in accurate and comprehensive single-cell analyses.

### Exemplary Chemistries for Generating MBIs Include, but are not Limited to

Thymine Glycol Formation (T to G Conversion): Thymine can be oxidized to form thymine glycol, a process that can be induced by chemical agents or radiation. During DNA replication or reverse transcription, thymine glycol can preferentially pair with a guanine (G), leading to a T-to-G transformation in the daughter strand.
8-Oxoguanine Formation (G to T Conversion): Guanine is susceptible to oxidation, forming 8-oxoguanine (8-oxo-G). This modified base can mispair with adenine (A) during DNA replication or reverse transcription, leading to a G-to-T transformation in the cDNA.
Alkylation of Guanine (G to A Conversion): Certain alkylating agents can modify the O6 position of guanine, leading to mispairing with thymine during replication or reverse transcription. This can result in a G-to-A transformation.
Uracil in DNA (Originally from Thymine Deamination): Thymine can be deaminated to uracil, which pairs with adenine. If present in DNA and transcribed, this would result in an A in the corresponding RNA. During reverse transcription, the uracil would base-pair with adenine, resulting in a T-to-A transformation in the cDNA.
Deamination (C to U Conversion, leading to C to T): Spontaneous or enzyme-catalyzed deamination of cytosine produces uracil (U), which can be recognized as thymine (T) during reverse transcription. So, during PCR amplification, a C-to-T conversion would be observed.
Hydroxymethylation and Further Oxidation (C to T Conversion): Cytosine can be hydroxymethylated to 5-hydroxymethylcytosine (5hmC), and subsequent oxidation can form 5-formylcytosine (5fC) and 5-carboxylcytosine (5caC). Certain DNA polymerases may misread these as thymines, resulting in C-to-T conversion during replication or reverse transcription.
Deamination (A to I Conversion, leading to A to G): Adenine can be deaminated to form inosine (I), which preferentially base-pairs with cytosine (C). During reverse transcription, this can lead to the incorporation of guanine (G) in the cDNA, resulting in an A-to-G conversion.
Alkylation (A to T or C Conversion): Certain alkylating agents can modify adenine in a way that leads to mispairing with thymine (T) or cytosine (C) during replication or reverse transcription. This would result in A-to-T or A-to-C transformation.
Hypoxanthine Formation (A to G Conversion): Adenine can be deaminated to hypoxanthine, which can base-pair with cytosine. During reverse transcription, this can lead to the incorporation of guanine, resulting in an A-to-G conversion.

Other modification methods that can modify a base in such a way that it is transformed into a different base, and this change can be detected by DNA sequencing.
Bisulfite Conversion: As previously mentioned, this can convert unmethylated cytosines into uracils, leading to a cytosine to thymine transformation upon PCR amplification.
Enzymatic Deamination: Adenosine deaminase can convert adenosines to inosines. Inosines are often read as guanines during sequencing, resulting in an adenine to guanine transformation.
Use of Modified Nucleotides that Pair Differently: Some modified nucleotides can pair with a different base during replication or reverse transcription. For example, 5-bromouracil (5-BU) can tautomerize and pair with guanine, potentially leading to thymine to cytosine transitions.
Chemical Modification Followed by Specific Enzymatic Conversion: Certain chemical reactions could modify a base in such a way that it is then recognized as a different base by a specific enzyme. This could result in a sequence change that is detectable by standard sequencing methods.
APOBEC/Cas9-Mediated Base Editing: Although mostly used for genome editing, APOBEC enzymes can deaminate cytosines, leading to uracils, which are read as thymines. This could be adapted for specific transformations in cDNA or RNA.
Incorporation of Unnatural Bases that are Processed as Natural Ones During Replication: Some synthetic bases can be incorporated into DNA or RNA and then processed as natural bases during subsequent replication or reverse transcription steps, leading to detectable changes in the sequence.

In some embodiments, random tagmentation, or another method of inserting DNA, is employed to generate non-natural sequences in a target nucleic acid. Semi-random deletions can also be used.

In some embodiments, a dedicated tag is added to target sequences of interest and modifications are made in the tag to generate diversity. This provides the advantage of preserving the original, unmodified sequence information in the remaining molecules.

By using one or more MBI approaches, the diversity of unique sequences readily exceeds that achieved by traditional UMI technologies.

Nucleic acid may be detected using a variety of techniques including but not limited to: nucleic acid sequencing, nucleic acid hybridization, and nucleic acid amplification.

Suitable nucleic acid sequencing techniques include, but are not limited to, sequencing by synthesis (see e.g., Meyer and Kircher, “Illumina sequencing library preparation for highly multiplexed target capture and sequencing,” Cold Spring Harbor Protocols 2010 (6)); single-molecule real-time sequencing (see e.g., Levene et al., “Zero-Mode Waveguides for Single-Molecule Analysis at High Concentrations,” Science. 299 (5607): 682-6 (2003)); ion semiconductor sequencing (see e.g., Rusk, “Torrents of sequence,” Nat. Methods 8, 44 (2011)); pyrosequencing (see e.g., Wicker et al., “454 sequencing put to the test using the complex genome of barley,” BMC Genomics, 7:275, 2006); sequencing by ligation (SOLID sequencing) (see e.g., Margulies et al., “Genome sequencing in microfabricated high-density picolitre reactors,” Nature, 437:376-80 (2005)); nanopore sequencing (see e.g., Goodwin et al., “Oxford Nanopore sequencing, hybrid error correction, and de novo assembly of a eukaryotic genome,” Genome Res., 25 (11): 1750-6 (2015)); chain termination sequencing (Sanger sequencing) (see e.g., Sanger et al., ″DNA sequencing with chain-terminating inhibitors, ″Proceedings of the National Academy of Sciences of the United States of America, 74 (12): 5463-5467 (1977)); and sequencing with mass spectrometry (see e.g., Edwards et al., “Mass-spectrometry DNA sequencing,” Mutation Research, 573 (1-2): 3-12 (2005)).

In some embodiments, a computer-based analysis program is used to translate the raw data generated by the detection assay (e.g., the presence, absence, or amount of a given marker or markers) into data of predictive value for a researcher or clinician. The researcher or clinician can access the predictive data using any suitable means. Thus, in some embodiments, the present invention provides the further benefit that the researcher or clinician, who may not be trained in genetics or molecular biology, need not understand the raw data. The data is presented directly to the researcher or clinician in its most useful form. The researcher or clinician is then able to immediately utilize the information (e.g., in order to optimize the care of a patient).

The present invention contemplates any method capable of receiving, processing, and transmitting the information to and from laboratories conducting the assays, information provider, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate data. Where the sample comprises a tissue or other biological sample, the subject may visit a medical center to have the sample obtained and sent to the profiling center, or subjects may collect the sample themselves (e.g., a saliva, stool, nasal swab, or urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced, specific for the diagnostic or prognostic information desired for the subject.

The profile data is then prepared in a format suitable for interpretation by a user (e.g., treating clinician). For example, rather than providing raw genetic data, the prepared format may represent a diagnosis or risk assessment (e.g., presence or absence of one or more biomarkers and the potential consequences thereof) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a phone, computer monitor, or other device.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may choose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

In some embodiments, the technology described herein is associated with a programmable machine designed to perform a sequence of arithmetic or logical operations as provided by the methods described herein. For example, some embodiments of the technology are associated with (e.g., implemented in) computer software and/or computer hardware. In one aspect, the technology relates to a computer comprising a form of memory, an element for performing arithmetic and logical operations, and a processing element (e.g., a microprocessor) for executing a series of instructions (e.g., a method as provided herein) to read, manipulate, and store data. In some embodiments, a microprocessor is part of a system for determining the presence or absence of a biomarker in a sample and for confirming that the biomarker is genuine and not the result of errors introduced by a biological assay (e.g., nucleic acid amplification) performed on the sample.

In some embodiments, the various embodiments of the present disclosure are associated with a plurality of programmable devices that operate in concert to perform a method as described herein. For example, in some embodiments, a plurality of computers (e.g., connected by a network) may work in parallel to collect and process data, e.g., in an implementation of cluster computing or grid computing or some other distributed computer architecture that relies on complete computers (with onboard CPUs, storage, power supplies, network interfaces, etc.) connected to a network (private, public, or the internet) by a conventional network interface, such as Ethernet, fiber optic, or by a wireless network technology.

For example, some embodiments provide a computer that includes a computer-readable medium. The embodiment includes a random access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, California and Motorola Corporation of Schaumburg, Illinois. Such processors include, or may be in communication with, media, for example computer-readable media, which stores instructions that, when executed by the processor, cause the processor to perform the steps described herein.

Computers are connected in some embodiments to a network. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers related to aspects of the technology provided herein may be any type of processor-based platform that operates on any operating system, such as Microsoft Windows, Linux, UNIX, Mac OS X, etc., capable of supporting one or more programs comprising the technology provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device. All such components, computers, and systems described herein as associated with the technology may be logical or virtual.

### DEFINITIONS

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The transitional phrase “consisting essentially of” as used in claims in the present application limits the scope of a claim to the specified materials or steps “and those that do not materially affect the basic and novel characteristic(s)” of the claimed invention, as discussed in In re Herz, 537 F.2d 549, 551-52, 190 USPQ 461, 463 (CCPA 1976). For example, a composition “consisting essentially of” recited elements may contain an unrecited contaminant at a level such that, though present, the contaminant does not alter the function of the recited composition as compared to a pure composition, i.e., a composition “consisting of” the recited components.

The term “one or more,” as used herein, refers to a number higher than one. For example, the term “one or more” encompasses any of the following: two or more, three or more, four or more, five or more, six or more, seven or more, eight or more, nine or more, ten or more, twelve or more, thirteen or more, fourteen or more, fifteen or more, twenty or more, fifty or more, 100 or more, or an even greater number.

As used herein, the term “sample” or “biological sample” encompasses a variety of sample types obtained from a variety of sources, which sample types contain biological material. For example, the term includes biological samples obtained from a mammalian subject, e.g., a human subject, and biological samples obtained from a food, water, or other environmental source, etc. The definition encompasses blood and other liquid samples of biological origin, as well as solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “sample” or “biological sample” encompasses a clinical sample, and also includes cells in culture, cell supernatants, cell lysates, cells, serum, plasma, biological fluid, and tissue samples.

The term “next generation sequencing” refers to highly parallelized methods of performing nucleic acid sequencing and comprises the sequencing-by-synthesis or sequencing-by-ligation platforms (e.g., employed by Illumina, Life Technologies, Pacific Biosciences and Roche, etc.). Next generation sequencing methods may also include, but not be limited to, nanopore sequencing methods such as offered by Oxford Nanopore or electronic detection-based methods such as the Ion Torrent technology commercialized by Life Technologies.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. Examples of non-isolated nucleic acids include a given DNA sequence (e.g., a gene) found on the host cell chromosome in proximity to neighboring genes; RNA sequences, such as a specific mRNA sequence encoding a specific protein, found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may be single-stranded), but may contain both the sense and anti-sense strands (i.e., the oligonucleotide may be double-stranded). An isolated nucleic acid may, after isolation from its natural or typical environment, be combined with other nucleic acids or molecules. For example, an isolated nucleic acid may be present in a host cell into which it has been placed, e.g., for heterologous expression.

The term “purified” refers to molecules, either nucleic acid or amino acid sequences that are removed from their natural environment, isolated, or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the terms “purified” or “to purify” also refer to the removal of contaminants from a sample. The removal of contaminating proteins results in an increase in the percent of polypeptide or nucleic acid of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

As used herein, the terms “patient” or “subject” refer to organisms to be subject to various tests described herein. The term “subject” includes animals, preferably mammals, including humans. In a preferred embodiment, the subject is a primate. In an even more preferred embodiment, the subject is a human. Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary therapeutic uses are provided herein. As such, the present disclosure provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to carnivores such as cats and dogs; swine, including pigs, hogs, and wild boars; ruminants and/or ungulates such as cattle, oxen, sheep, giraffes, deer, goats, bison, and camels; pinnipeds; and horses. Thus, also provided is the diagnosis and treatment of livestock, including, but not limited to, domesticated swine, ruminants, ungulates, horses (including racehorses), and the like.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, percentage methylation, allele frequencies, RNA expression levels, protein expression, phenotypes correlating to genotypes, etc.

What is claimed:

1. A method for identifying a target nucleic acid molecule using modified base identifiers (MBIs) comprising:
a) modifying a molecule or its copy with base modifications, wherein a generated pattern of modified bases are unique or substantially unique to the molecule; and
b) reading the modified molecule, or a copy of part or all thereof, and determining the locations and/or types of modified bases to differentiate the modified molecule from other molecules.

2. A method of claim 1, wherein the target nucleic acid is RNA and the modifying comprises reverse transcription.

3. A method of claim 1, wherein the base modifications comprise methylated nucleotides.

4. A method of claim 1, wherein frequency of base modification is controlled by controlling a ratio of modified to non-modified base concentrations during a copying step within the modifying step.

5. A method of claim 1, where in the target further comprises or receives a tag sequence comprising a barcodes or unique molecular identifier (UMI).

6. A method of claim 1, wherein in the base modifications comprise one or more of Thymine Glycol Formation (T to G Conversion), 8-Oxoguanine Formation (G to T Conversion), Alkylation of Guanine (G to A Conversion), Uracil in DNA (Originally from Thymine Deamination or incorporated as a base), Hydroxymethylation and Further Oxidation (C to T Conversion), Deamination (C to U Conversion, leading to C to T), or Hypoxanthine Formation (A to G Conversion).

7. A method of claim 1, wherein said modifying comprises use of error prone enzymes, non-canonical nucleotides, polymerase inhibitors, or altering reaction conditions to generate randomized base modifications.

8. A method of claim 1, wherein said reading comprises sequencing the modified molecule and determining locations of modified bases in sequence reads by comparing a sequence read with sequence reads of similar sequence and identifying locations of known conversions.

9. The method of claim 8, wherein sequence reads of are grouped such that all reads for a given original target are used to generate a consensus sequence that uniquely differentiates the consensus sequence from all other original target molecules of identical starting sequence.

10. The method of claim 1, wherein identified modified bases are used to enumerate a number of original target molecules in a sample and/or correct for reaction biases.

11. The method of claim 1, wherein identified modified bases are used, in conjunction with an index or barcode sequence, to perform quantitative single cell gene expression profiling.

12. The method of claim 1, wherein said reading comprises duplex sequencing.

13. The method of claim 1, wherein said reading comprises building a consensus sequence for error correction.

14. A method for introducing modified base identifiers, comprising:
a) reacting a target molecule with a reactant that introduces at least semi-random base modifications into the target or copies of the target; and
b) using the base modifications to differentiate targets of otherwise identical sequence.

15. The method of claim 14, wherein the reacting comprises use of a methylase enzyme that randomly methylates bases in the target, thereby generating a signature of random base modifications.

16. A method for quantitative single cell gene expression profiling using modified base identifiers (MBIs) comprising:
a) creating a cDNA copy of mRNA by reverse transcription, in which methylated cytosines are included in the reaction at a fixed proportion to unmethylated cytosines, thereby generating for each cDNA of each mRNA molecule a unique methylated cytosine spectrum;
b) using bisulfate conversion to transform the unmethylated cytosines to uracils, leaving the methylated cytosines unchanged;
c) copying the modified cDNA such that Uracils are replaced with Thymines to generate modified copies;
d) sequencing the modified copies to generate sequence reads;
e) clustering the sequence reads based on their sequence; and
f) using locations of the Thymines and Cytosines to differentiate mRNA molecules of original identical sequence.

17. A method for sequencing long molecules, comprising:
a) labeling long nucleic acid molecules with modified base identifiers (MBIs) to generated labeled molecules;
b) amplifying the labeled molecules, preserving the MBIs in some of the copies;
c) fragmenting and sequencing the amplicon fragments, such that MBIs remain in the fragments and, in some case, the same MBI region exists within different fragments; and
d) using similarity of the MBI regions in different fragments to associate the fragments together to generate a longer read.

18. A method for sequencing single cells at high throughput, comprising:
a) labeling nucleic acids of at least one cell with a tag sequence relating the cell identity;
b) labeling the nucleic acids of said cell with MBIs so as to differentiate between nucleic acids of otherwise identical sequence from said cell; and
c) analyzing the tag, nucleic acid sequence, and MBI so as to accurately identify the nucleic acids of single cells.

19. The method of claim 18, wherein the cells are in partitions and the tag sequences relating cell identity are introduced by combinatorial labeling using permeabilization to facilitate incorporation through cell membranes.

20. The method of claim 19, wherein the cells are partitioned, such that the tag sequence is substantially unique to the partitions.
